(19) United States
(12) Patent Application Publication
(10) Pub. No.: US 2020/0084869 A1
(43) Pub. Date: Mar. 12, 2020

(54) MOBILE HIGH VOLTAGE GENERATING DEVICE

(71) Applicant: Delta Electronics,Inc., Taoyuan City (CN)

(72) Inventors: Hongyuan JIN, Taoyuan City (CN); Yao TAO, Taoyuan City (CN); Liangyuan PU, Taoyuan City (CN); Xintian LI, Taoyuan City (CN); Dezhi JIAO, Taoyuan City (CN)

(73) Assignee: Delta Electronics,Inc., Taoyuan City (CN)

(21) Appl. No.: 16/513,931

(22) Filed: Jul. 17, 2019

(30) Foreign Application Priority Data
Sep. 7, 2018 (CN) 201821468992.0

(51) Int. Cl. H05G 1/12; H02M 7/219; H02M 7/521; H02J 7/00; A61B 6/00; H02M 3/335

(52) U.S. Cl. CPC H05G 1/12; H02M 7/219; H02M 7/521; A61B 2560/0214; A61B 6/56; H02M 3/33576; H02J 7/0052

(57) ABSTRACT

A mobile high voltage generating device for providing power to an X-ray apparatus, includes a power supply unit including a battery and an isolated DC-DC converter electrically coupled with the battery; an energy storage unit, electrically coupled to the isolated DC-DC converter of the power supply d a voltage conversion unit coupled between the energy storage unit and a radiation source of the X-ray apparatus; wherein the isolated DC-DC converter includes: a first inverter circuit coupled to a battery, a first rectifier circuit coupled to the energy storage unit, a first transformer including a primary winding and a secondary winding. The primary winding is coupled to the first inverter circuit and the secondary winding is coupled to the first rectifier circuit.

### CROSS REFERENCE

This application is based upon and claims the priority of Chinese Patent Applications No. 201821468992.0, filed on Sep. 7, 2018, the entire contents thereof are incorporated herein by reference.

### TECHNICAL FIELD

The present disclosure relates to a high voltage generating device, in particular, to a mobile high voltage generating device applied on an X-ray apparatus.

### BACKGROUND

The mobile X-ray apparatus has been applied to outdoor diagnosis, disaster relief and other occasions, and in these occasions, X-ray images can be simply and conveniently obtained by X-ray apparatus to diagnose the state of patients or animals. The mobile X-ray apparatus is generally powered directly by the battery, and the X-ray emitting energy is directly provided by the battery. When the X-ray apparatus is emitting, the battery of the mobile X-ray apparatus is in a state of high current discharge, which reduces the capacity utilization of the battery and shortens the service life of the battery.

### SUMMARY

One aspect of the present disclosure is provided a mobile high voltage generating device for providing power to an X-ray apparatus, including: a power supply unit comprising a battery and an isolated DC-DC converter electrically coupled with the battery; an energy storage unit, electrically coupled to the isolated DC-DC converter of the power supply unit; a voltage conversion unit, electrically coupled to the energy storage unit and a radiation source of the X-ray apparatus; wherein the isolated DC-DC converter further includes a first inverter circuit, a first transformer having a primary winding and a secondary winding, and a first rectifier circuit; wherein the first inverter circuit is electrically coupled to the battery, and the primary winding of the first transformer is electrically coupled to the first inverter circuit, and the secondary winding of the first transformer is electrically coupled to the first rectifier circuit, and the first rectifier circuit is electrically coupled to the energy storage unit.

In order to further understand the features and technical contents of the present disclosure please refer to the following detailed description and drawings related to the present disclosure. However, the detailed description and the drawings are merely illustrative of the disclosure and are not intended to limit the scope of the claims.

### BRIEF DESCRIPTION OF THE DRAWINGS

Embodiments of the present disclosure will now be described, by way of example only, with reference to the accompanying schematic drawings in which corresponding reference symbols indicate corresponding parts, and in which:

FIG. 1 shows a schematic view of a block diagram of a mobile high voltage generating device of one embodiment of the present disclosure;

FIG. 2 shows a schematic view of a block diagram of an isolated direct current-direct current (DC-DC) converter of one embodiment of the present disclosure;

FIG. 3 shows circuits of a block diagram of an isolated DC-DC converter of one embodiment of the present disclosure;

FIG. 4 shows circuits of a block diagram of an isolated DC-DC converter of one embodiment of the present disclosure;

FIG. 5 shows circuits of a block diagram of an isolated DC-DC converter of one embodiment of the present disclosure;

FIG. 6 shows a schematic diagram of circuits of an energy storage unit of one embodiment of the present disclosure;

FIG. 7 shows a schematic diagram of circuits of an energy storage unit of one embodiment of the present disclosure;

FIG. 8 shows a schematic diagram of circuits of an energy storage unit of one embodiment of the present disclosure;

FIG. 9 shows a schematic diagram of a discharging current of a battery and an input voltage of a radiation source of a mobile X-ray apparatus in prior art; and

FIG. 10 shows a schematic diagram of a discharging current of the battery of a mobile high voltage generating device and an input voltage of a radiation source of the present disclosure.

### DETAILED DESCRIPTION

In the following, embodiments of the present disclosure will be described in detail referring to figures. The concept and its realizations of the present disclosure can be implemented in a plurality of forms, and should not be understood to be limited to the embodiments described hereafter. In contrary, these embodiments are provided to make the present disclosure more comprehensive and understandable, and so the conception of the embodiments can be conveyed to the technicians in the art fully. Same reference signs in the figures refer to same or similar structures, so repeated description of them will be omitted.

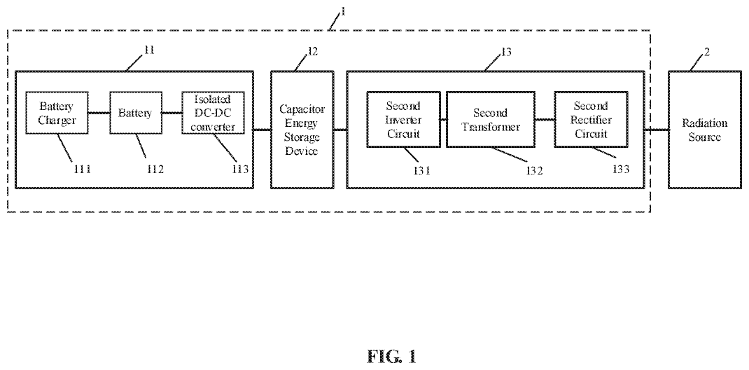

FIG. 1 shows a schematic view of a block diagram of a mobile high voltage generating device 1 of one embodiment of the present disclosure. As shown in FIG. 1, the high voltage generating device 1 is coupled to a radiation source 2 of an X-ray apparatus. The high voltage generating device 1 is configured to provide power to the radiation source 2. In this embodiment, the mobile high voltage generating device 1 includes a power supply unit 11, an energy storage unit 12 and a voltage conversion unit 13. The power supply unit 11 provides an intermediate voltage to charge the energy storage unit 12. In this embodiment, when the X-ray apparatus is activated to emit the X-ray, the voltage conversion unit 13 is powered by the energy storage unit 12, which boosts the voltage on the energy storage unit 12 and powers the radiation source 2 coupled with the voltage conversion unit 13.

Referring to FIG. 1, the power supply unit 11 includes a battery 112 and an isolated DC-DC converter 113. The battery 112 provides a first voltage to the DC-DC converter 113. The DC-DC converter 113 generates the intermediate voltage according to the first voltage to charge the energy storage unit L. Wherein, the voltage of the battery is less than or equal to 60V In some embodiments, the power supply unit 11 also includes a battery charger 111, the battery charger 111 is coupled with the battery 112. In some embodiments, the battery charger 111 is coupled to an external power, e.g. a power grid, a battery and a power generator, to charge the battery 112 of the power supply unit 11. According to TEC 60601-1 of international standard, for accessible parts including applied parts, the voltage to earth or to other accessible parts shall not exceed 42, 4V peak a.c. or 60V d.c. in normal condition or in single fault condition. The d.c. limit of 60V applies to d.c. with not more than 10% peak-to-peak ripple. So the limitation of the voltage of the battery 112 not only meets the requirements of the international standard, but also can ensure the safety and stability of the system.

As shown in FIG. 1, the voltage conversion unit 13 includes a second inverter circuit 131, a second transformer 132 having a primary winding and a secondary winding and a second rectifier circuit 133. Wherein the second inverter circuit is coupled to the energy storage unit 12. The primary winding of the second transformer 132 is electrically coupled to the second inverter circuit 131, and the secondary winding of the second transformer 132 is electrically coupled to he second rectifier circuit 133. And the second rectifier circuit 133 is coupled to the radiation source 2 of the X-ray apparatus. The second inverter circuit 131 is configured to convert a DC current received from the energy storage unit 12 to a high frequency AC current to the second transformer 132. And the high frequency AC current is then converted to a DC current by the second rectifier circuit 133. The DC current is then provided to the radiation source 2 of the X-ray apparatus. An X-ray is then generated by the radiation source 2 powered by the voltage conversion unit 13. In generally, the range of the output voltage of the voltage conversion unit is 40KV to 150KV. During the process of generating the X-ray by the radiation source 2, the output voltage of the voltage conversion unit substantially maintains constant which means the value of the output voltage of the voltage conversion unit 13 during the X-ray emitting time process is substantially constant.

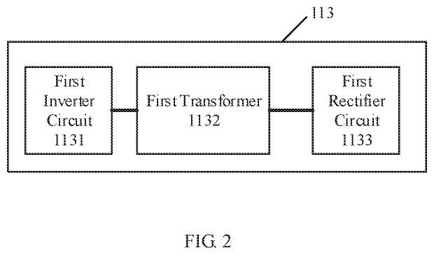

FIG. 2 shows a schematic view of a block diagram of the isolated DC-DC converter 113 of one embodiment of the present disclosure. In this embodiment, the isolated DC-DC converter 113 includes a. first inverter circuit 1131, a first transformer 1132 having a primary winding and a secondary winding, and a first rectifier circuit 1133. Wherein, the first inverter circuit 1131 is coupled to the battery 112, the primary winding of the first transformer 1132 is electrically coupled to the first inverter circuit 1131, and the secondary winding of the first transformer 1132 is electrically coupled to the first rectifier circuit 1133, and a first rectifier circuit 1133 is electrically coupled to the energy storage unit 12. The first inverter circuit 1131. in some embodiments, includes a full bridge circuit. In some embodiments, the first inverter circuit 1131 includes a half bridge circuit. The first rectifier circuit 1133, in some embodiments, includes a full bridge rectifier circuit. In some embodiments, the first rectifier circuit 1133 includes a full wave rectifier circuit. Wherein, the output voltage of the first rectifier circuit 1133 is the intermediate voltage. In some embodiments, the isolated DC-DC converter is a regulated voltage converter, and the output voltage of the isolated DC-DC converter 113 is controlled to be stable, so the output voltage of the isolated DC-DC converter 113 won't be changed with voltage of the battery 112. In some embodiments, the isolated DC-DC converter is an unregulated voltage converter, and the output voltage of the isolated DC-DC converter 113 is not controlled, so the output voltage of the isolated DC-DC converter 113 may vary with the change of the voltage of the battery 112. Further, a resonant circuit is coupled between the first inverter circuit and the first transformer. In some embodiments, the resonant circuit includes an inductor. In some embodiments, the resonant circuit includes at least one inductor and at least one capacitor.

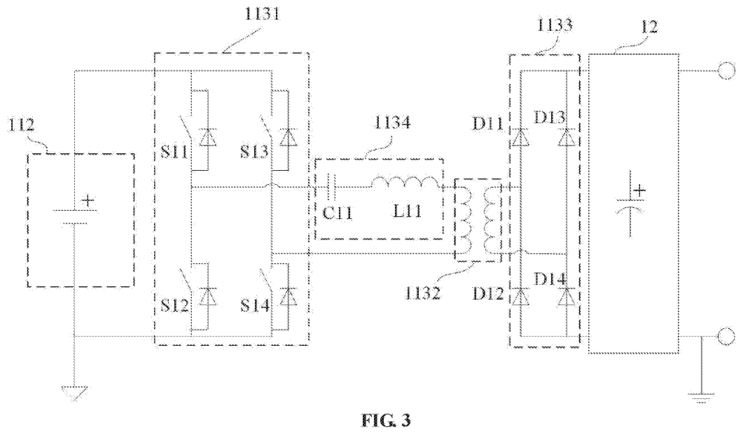

FIG. 3 shows circuits of a block diagram of an isolated DC-DC converter of one embodiment of the present disclosure. As shown in FIG. 3, the first inverter circuit 1131 is a full bridge circuit. The full bridge circuit includes a first bridge arm and a second bridge arm. The first bridge arm includes a first switch S11 and a second switch S12 connected in series with each other. The second bridge arm is connected in parallel with the first bridge arm. The second bridge arm includes a third switch 513 and a fourth switch S14 connected in series with each other. A resonant circuit 1134 is coupled between the first inverter circuit 1131 and the first transformer 1132. In this embodiment, the resonant circuit 1134 includes a resonant capacitor C11 serially coupled to a resonant inductance L11. A common node of the first switch S11 and the second switch S12 is electrically coupled, via the resonant circuit 1134, to a first end of the primary winding of the first transformer 1132. A common node of the third switch S13 and the fourth switch S14 is coupled to a second end of the primary winding of the first transformer 1132. As shown in FIG. 3, the first rectifier circuit 1133 is a full bridge rectifier circuit. The full bridge rectifier circuit includes a first diode D11 serially connected with a second diode D12 and a third diode D13 serially connected with a fourth diode D14. A common node of the first diode D11 and the second diode D12 is coupled to a first end of the secondary winding of the first transformer 1132. A common node of the third diode D13 and the fourth diode D14 is coupled to a second end of the secondary winding of the first transformer 1132.

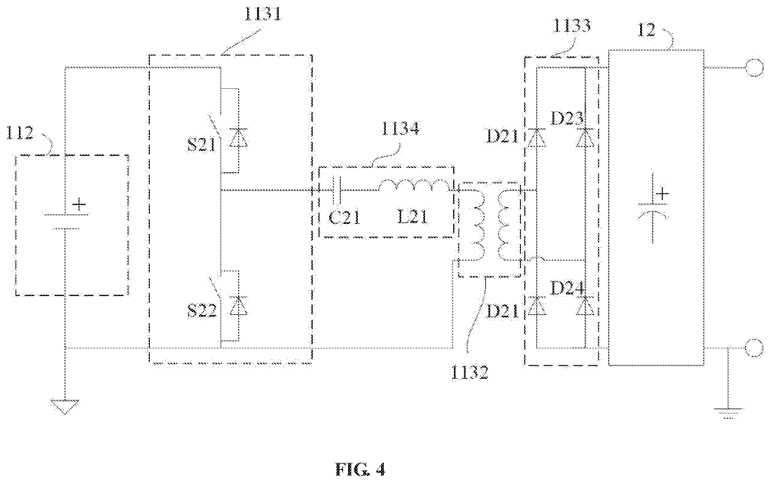

FIG. 4 shows circuits of a block diagram of an isolated DC-DC converter 113 of one embodiment of the present disclosure. The first inverter circuit 1131 is a half bridge circuit. The first inverter circuit 1131 includes a first bridge arm having a first switch S21 serially connected with a second switch S22. A common node of the first switch S21 and the second switch S22 is electrically coupled, via the resonant circuit 1134, to a first end of the primary winding of the first transformer 1132. The second end of the primary winding of the first transformer 1132 is electrically coupled to the second switch S22. The resonant circuit 1134 includes a resonant capacitor C21 and a resonant inductance L21. As shown in FIG. 4, the first rectifier circuit 1133 is a full bridge rectifier circuit. The full bridge rectifier circuit includes a first diode D21 serially coupled to a second diode D22 and a third diode D23 serially coupled to a fourth diode D24. A common node of the first diode D21 and the second diode D22 is coupled to a first end of the secondary winding of the first transformer 1132. A common node of the third diode D23 and the fourth diode D24 is coupled to a second end of the secondary winding of the first transformer 1132.

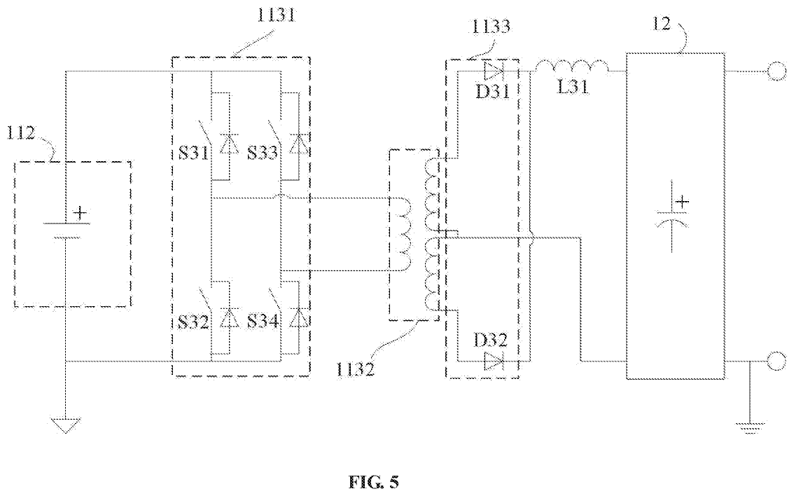

FIG. 5 shows circuits of a block diagram of an isolated DC-DC converter 113 of one embodiment of the present disclosure. As shown in FIG. 5, in this embodiment, the first inverter circuit 1131 is a full bridge circuit. The first rectifier circuit 1133 is a full wave rectifier circuit. The first inverter circuit 1131 includes a first bridge arm and a second bridge arm connected in parallel with the first bridge arm. The first bridge arm includes a first switch S31 and a second switch S32. A common node of the first switch S31 and the second switch S32 is electrically coupled to a first end of the primary winding of the first transformer 1132. The second bridge arm includes a third switch S33 serially coupled to a fourth switch S34. A common node of the third switch S33 and the fourth switch S34 is electrically coupled to a second end of the primary winding of the first transformer 1132. The first rectifier circuit 1133 includes a first diode D31, a second diode D32 and an inductance L31. A first end of the inductance L31 is coupled to the cathode of the first diode D31 and the cathode of the second diode D32. The secondary winding of the first transformer 1132 is coupled between the anode of the first diode D31 and the anode of the second diode D32. A second end of the inductance L31 and the center tap of the secondary winding of the first transformer 1132 are coupled to an input end of the energy storage unit

In some embodiments, the energy storage unit 12 includes a super capacitor. In some embodiments, the energy storage unit 12 includes an electrolytic capacitor.

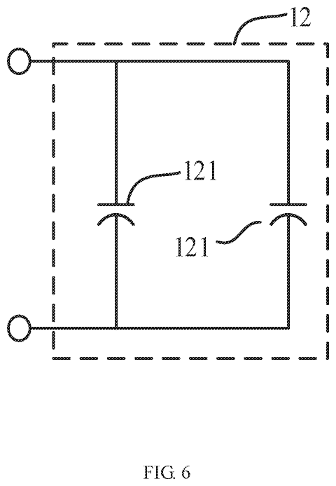

FIG. 6 shows a schematic diagram of circuits of the energy storage unit 12 of one embodiment of the present disclosure. As shown in FIG. 6, the energy storage unit 12 includes two storage capacitors 121 connected in parallel. In some embodiments, the energy storage unit 12 includes a plurality of storage capacitors 121 connected in parallel.

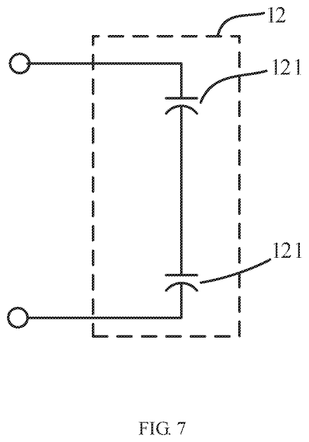

FIG. 7 shows a schematic diagram of circuits of an energy storage unit 12 of one embodiment of the present disclosure. As shown in FIG. 7, the energy storage unit 12 includes two storage capacitors 121 connected in series. In some embodiments, the energy storage unit 12 includes a plurality of storage capacitors 121 connected in parallel.

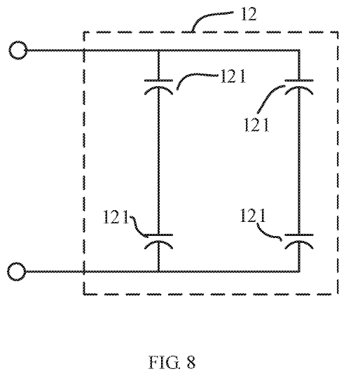

FIG. 8 shows a schematic diagram of circuits of an energy storage unit 12 of one embodiment of the present disclosure. As shown in FIG. 8, the energy storage unit 12 includes four storage capacitors 121. Each two of the storage capacitors 121 is connected in series to form two paths. The two paths are then connected in parallel. Further, the energy storage unit 12 includes a plurality of storage capacitors 121, some of the storage capacitors 121 are connected in series, and some storage capacitors 121 are connected in parallel. That is to say, the storage capacitors 121 can be arranged in series and parallel according to actual needs.

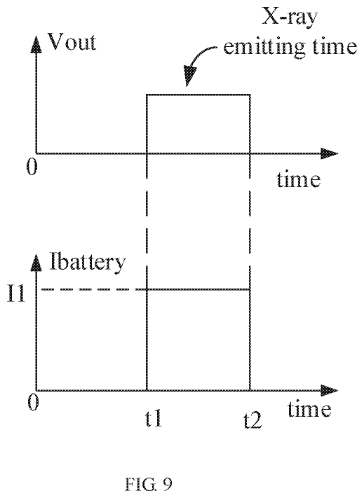

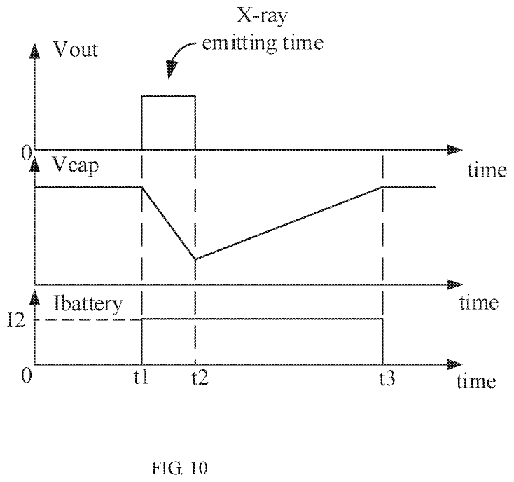

FIG. 9 shows a schematic diagram of a discharging current of a battery and an input voltage of a radiation source of a mobile X-ray apparatus in prior art. FIG. 10 shows a schematic diagram of a discharging current of the battery of a mobile high voltage generating device 1 and an input voltage of a radiation source 2 of the present disclosure. Wherein, the need of the input voltage of the radiation source of the mobile X-ray apparatus in prior art is for example, 150 kV. And the need of the input voltage of the radiation source 2 of the mobile X-ray apparatus in one embodiment of the present disclosure is also 150 kV. It should be noted, the input voltage of the radiation source 2 of the mobile X-ray apparatus can be set other values, for example, in a range of 40kV-150kV. Meanwhile, for comparison, the mobile X-ray apparatus in one embodiment of the present disclosure and the mobile X-ray apparatus in prior art choose the same batteries. Wherein, the prior art of the mobile X-ray apparatus does not include the capacitor energy storage unit

As shown in FIG. 9 and FIG. 10, during the X-ray emitting time, from time t1 to t2, a discharging current I1 of the battery of the prior art is greater than a discharging current t2 of the battery of the present disclosure. Specifically, as shown in FIG. 9, the battery needs to be directly and fully discharged to power the radiation source. However, as shown in FIG. 10, before the X-ray is emitted, the energy storage unit 12 is charged by the battery, and the voltage Vcap of the energy storage unit 12 maintains needed voltage level. During the X-ray emitting time t1-t2, the energy of the energy storage unit 12 is released to directly provide energy to the radiation source, and the battery 112 powers energy storage unit 12. So the radiation source is not powered directly by the battery 112. When the X-ray emitting is end at the time t2, the voltage of the energy storage unit 12 is decreased to reach the lowest point. Further, during time t2 to t3, in this embodiment, the energy storage unit 12 continues to be charged by the battery 112 until the voltage Vcap of the energy storage unit 12 reaches the needed voltage gradually. As shown in FIG. 10, in this embodiment, the discharging current I2 of the battery, time t1 to t3, is smaller than the discharging current I1, time t1 to t2 shown in FIG. 9. of the prior art of the mobile X-ray apparatus.

And the discharging time t1 to t3 of the battery shown in FIG. 10, of this embodiment is greater than the discharging time t1 to t2, of the prior art of the mobile X-ray apparatus. According to the principle of energy conservation, under the circumstance of the same energy consumed by the X-ray apparatus of the present disclosure and the prior art, the discharging current I2 of the battery of the present disclosure is smaller than the discharging current I1 of the prior art. Therefore, the discharge C rate of the present disclosure is smaller than the discharge C rate of the prior art. The battery utilization rate of the present disclosure is better than the battery utilization rate of the prior art.

In summary, an energy storage unit is provided between the power supply unit and the voltage conversion unit in the mobile high-voltage generating device of the present disclosure, and the energy storage unit provides power to the voltage conversion unit instead of directly providing power to the voltage conversion unit by the battery, which reduces the discharge C rate of the battery.

The energy required for X-ray emitting is mainly from the storage capacitor unit, regardless of the discharge state of the battery. Therefore, it is flexible to select a battery with a suitable capacity according to the actual emitting endurance demand. The battery charges the energy storage unit through the isolated DC-DC converter, which isolates the battery from the energy storage unit, thereby making the system more secure and reliable.

While the disclosure has been described by way of example and in terms of the preferred embodiments, it is to be understood that the disclosure is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A mobile high voltage generating device for providing power to an X-ray apparatus, comprising:
a power supply unit comprising a battery and an isolated DC-DC converter electrically coupled with the battery;
an energy storage unit, electrically coupled to the isolated DC-DC converter of the power supply unit; and
a voltage conversion unit, electrically coupled to the energy storage unit and a radiation source of the X-ray apparatus.

2. The mobile high voltage generating device of claim 1, wherein the isolated DC-DC converter further comprises a first inverter circuit, a first transformer having a primary winding and a secondary winding, and a first rectifier circuit; wherein the first inverter circuit is electrically coupled to the battery, and the primary winding of the first transformer is electrically coupled to the first inverter circuit, and the secondary winding of the first transformer is electrically coupled to the first rectifier circuit, and the first rectifier circuit is electrically coupled to the energy storage unit.

3. The mobile high voltage generating device of claim 2, wherein the first inverter circuit is a full bridge circuit.

4. The mobile high voltage generating device of claim 3, wherein the full bridge circuit comprises:
a first bridge arm including a first switch and a second switch connected in series with each other, and a common node of the first switch and the second switch electrically coupled to a first end of the primary winding of the first transformer;
a second bridge arm connected in parallel with the first bridge arm, including a third switch and a fourth switch connected in series with each other, and a common node of the third switch and the fourth switch electrically coupled to a second end of the primary winding of the first transformer.

5. The mobile high voltage generating device of claim 2, wherein the first inverter circuit is a half bridge circuit.

6. The mobile high voltage generating device of claim 5, wherein the half bridge circuit comprises:
a first bridge arm including a first switch and a second switch connected in series with each other, and a common node of the first switch and the second switch electrically coupled to a first end of the primary winding of the first transformer, and a second end of the primary winding of the first transformer coupled to the second switch.

7. The mobile high voltage generating device of claim 2, wherein a resonant circuit is coupled between the first inverter circuit and the first transformer.

8. The mobile high voltage generating device of claim 7, wherein the resonant circuit comprises at least one inductor and at least one capacitor.

9. The mobile high voltage generating device of claim 2, wherein the first rectifier circuit is a full bridge rectifier circuit.

10. The mobile high voltage generating device of claim 2, wherein the first rectifier circuit is a full wave rectifier circuit.

11. The mobile high voltage generating device of claim 1, wherein the voltage conversion unit comprises a second inverter circuit, a second transformer having a primary winding and a secondary winding and a second rectifier circuit; wherein the second inverter circuit is electrically coupled to the energy storage unit, and the primary winding of the second transformer is electrically coupled to the second inverter circuit, and the secondary winding of the second transformer is electrically coupled to the second rectifier circuit, and the second rectifier circuit is electrically coupled to the radiation source.

12. The mobile high voltage generating device of claim 1, wherein the power storage unit comprises a plurality of capacitors connected in parallel.

13. The mobile high voltage generating device of claim 1, wherein the power storage unit comprises a plurality of capacitors connected in serial.

14. The mobile high voltage generating device of claim 1, wherein the power storage unit comprises a supper capacitor.

15. The mobile high voltage generating device of claim 1, wherein the power storage unit comprises an electrolytic capacitor.

16. The mobile high voltage generating device of claim 1, wherein the power supply device further comprises a battery charger configured to charge the battery.

17. The mobile high voltage generating device of claim 1, wherein the isolated DC-DC converter is a regulated voltage converter, or the isolated DC-DC converter is an unregulated voltage converter.

18. The mobile high voltage generating device of claim 1, wherein a voltage of the battery is equal to or less than 60V.
